(19) United States
(12) Patent Application Publication
(10) Pub. No.: US 2014/0066806 A1
(43) Pub. Date: Mar. 6, 2014

(54) Breast Biopsy Needle Localization Cover

(71) Applicant: Howato Industries, Manalapan, NJ (US)

(72) Inventor: Howato Industries, Manalapan, NJ (US)

(21) Appl. No.: 13/599,401

(22) Filed: Aug. 30, 2012

(51) Int. Cl. A61B 10/02

(52) U.S. Cl. 600/567

(57) ABSTRACT

What is disclosed is a medical device for covering the protruding end of a localization wire during a breast biopsy procedure. The cover is a generally hollow bell shaped device with an opening on the bottom thereof for receipt of the protruding end of the wire. In addition, there are adhesive tabs on the bottom of the cover to attach the device to the patient after placement over the protruding end of the wire.

### BACKGROUND OF THE INVENTION

The present invention is directed to the medical device field. In particular, the present invention is directed to use in connection with a breast biopsy procedure. Prior to a breast biopsy, a surgeon performs a localization procedure using a needle and/or a hook-wire. The purpose of the localization procedure is to place the needle or a hook-wire through or quite near to a mass, area of architectural distortion or suspicious calcification prior to surgery. The localization procedure is necessary because the surgeon cannot see or feel the area where the abnormality may be present but uses the localization needle and hook-wire as a guide to where to perform the surgery.

Once the breast lesion is localized, a portion of the needle and hook-wire are within the breast with the remainder protruding from the patient. The wire must remain in place during the biopsy procedure. Currently, the protruding portion of the wire is covered with a simple dressing, a mask or a small cup. The present methods are problematic in that each is cumbersome, not sterile and not uniform.

The primary object of the present invention is to solve the problems of the current methods for covering the protruding ends of the localization needles in breast biopsy procedures. Consequently, the present invention is directed to a simple easy to use sterile device that is intended to cover the protruding localization wire.

### SUMMARY OF THE INVENTION

A disposable breast biopsy needle localization cover comprising a generally hollow receiving device with a top end and a bottom end wherein the top end is closed and the bottom end is open; an adhesive device affixed to the receiving device wherein the receiving device is adapted to receive the end of the breast biopsy needle that protrudes from the patient and the adhesive device is used to removably attach the cover to the patient during the biopsy procedure.

### BRIEF DESCRIPTION OF THE DRAWINGS

FIG. 1 is a plan view of a device in accordance with the present invention in use.

FIG. 2 is a plan view of a device in accordance with the present invention.

FIG. 3 is a side elevation view of the device shown in FIG. 2.

FIG. 4 is a top elevation view of the device shown in FIG. 2.

FIG. 5 is a bottom plan view of the device shown in FIG. 2.

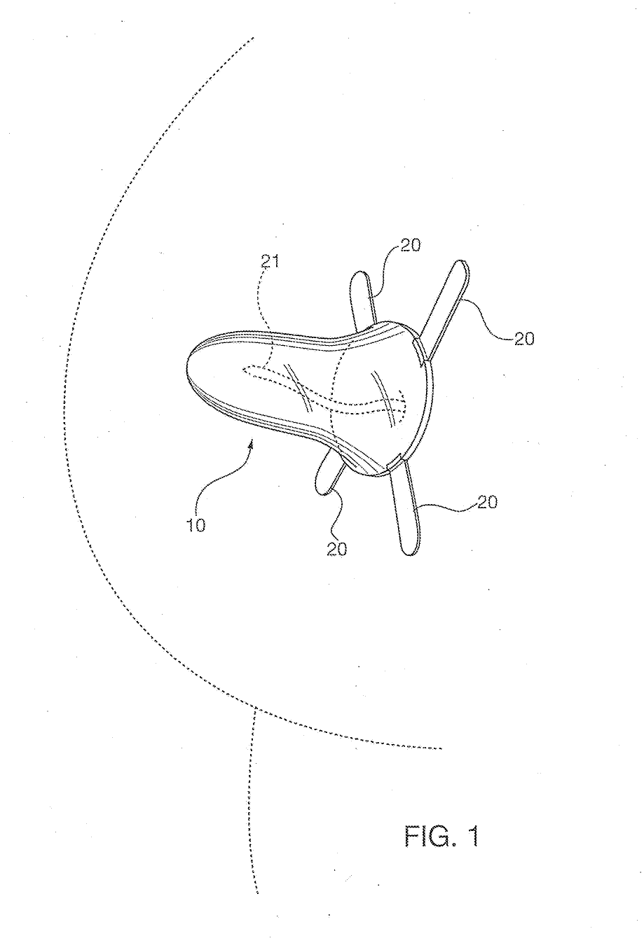

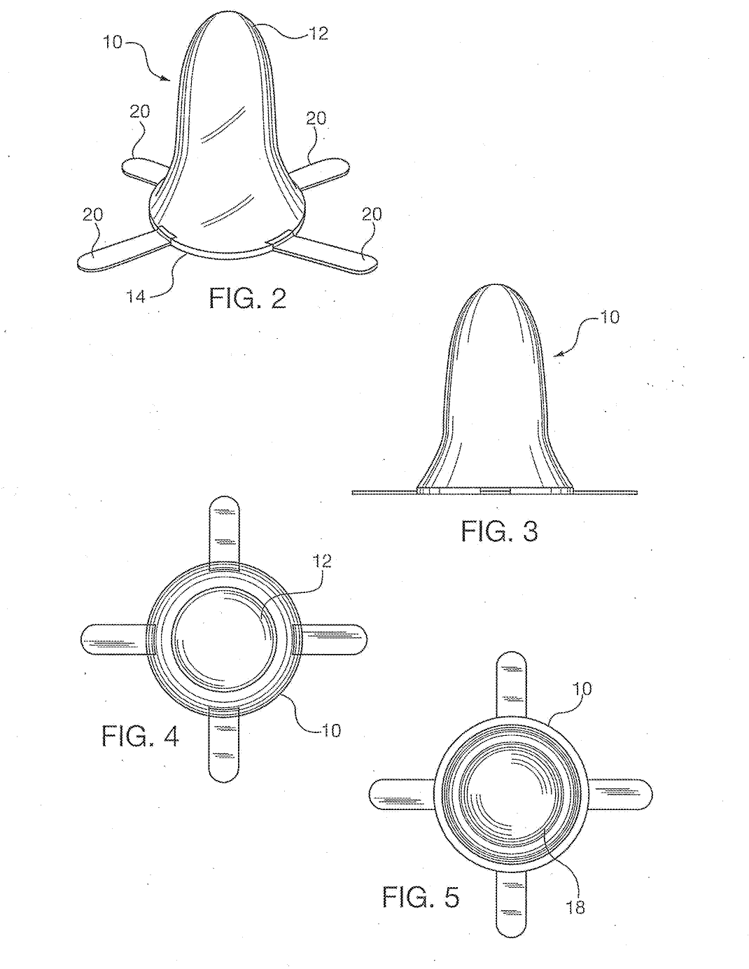

### DETAILED DESCRIPTION OF THE PRESENT INVENTION

The presently preferred embodiment of the present invention as illustrated in the drawings will now be described. Those of ordinary skill in the art will recognize that many obvious variations may be made to this embodiment without departing from the spirit or scope of the present invention.

The breast biopsy localization needle cover 10 is shown in the figures. The cover 10 is generally bell-shaped with a top end 12 and a bottom end 14. The cross-section of the bottom end 11 is generally larger than the cross-section near the top end 12. The cover 10 comprises an opening 18 at the bottom end 14. The cover 10 further comprises a plurality of adhesive tabs 20. In this embodiment, four adhesive tabs 20 are shown but that number can vary.

It is contemplated that the cover 10 can be fabricated from a lightweight yet durable plastic material. The material must be capable of being sterilized for medical use and shall maintain its size and shape. Further, the material must be strong enough not to collapse upon itself or to bend during use.

In practice, the cover 10 will be placed over the localization needle 21 protruding from the patient's skin. The protruding end of the localization needle 21 will be inserted into the opening 18. The bottom end 16 of the cover 10 will be placed in contact with the patient's skin and the cover held in place during the procedure by the adhesive tabs 20. At the end of the procedure, the cover 10 can be easily removed from the patient and discarded.

The dimensions of the device can be determined based upon the needle size and patent. However, this determination is within the skill of those of ordinary skill in the art.

What is claimed is:

1) A disposable breast biopsy needle localization cover comprising:
a) a generally hollow receiving device with a top end and a bottom end wherein the top end is closed and the bottom end is open;
b) an adhesive device affixed to the receiving device; and
wherein the receiving device is adapted to receive the end of the breast biopsy needle that protrudes from the patient and the adhesive device is used to removably attach the cover to the patient during the biopsy procedure.

2) The disposable breast biopsy localization cover of claim 1 wherein the cover is tapered with a large cross-section at the bottom end and a smaller cross-section at the top end.

3) The disposable breast biopsy needle localization cover of claim 2 wherein the cover is generally bell-shaped.

4) The disposable breast biopsy needle localization cover of claim 1 wherein the cover is fabricated from a light weight plastic material that is capable of being medically sterilized and still retain its size and shape.
